(19) United States
(12) Patent Application Publication
(10) Pub. No.: US 2021/0406700 A1
(43) Pub. Date: Dec. 30, 2021

(54) SYSTEMS AND METHODS FOR TEMPORALLY SENSITIVE CAUSAL HEURISTICS

(71) Applicant: KPN Innovations, LLC, Lakewood, CO (US)

(72) Inventor: Kenneth Neumann, Lakewood, CO (US)

(21) Appl. No.: 16/912,019

(22) Filed: Jun. 25, 2020

(51) Int. Cl. G06N 5/00; G06N 20/00; G06N 5/04

(52) U.S. Cl. CPC G06N 5/003; G06N 5/04; G06N 20/00

(57) ABSTRACT

A system for temporally sensitive causal heuristics, the system comprising a computing device includes a computing device configured to provide a plurality of constitutional events and a plurality of potential effects relating to a human subject, wherein each constitutional event of the plurality of constitutional events includes an event type, a significance level, a time of occurrence, a temporal function, and at least a potential effect of the plurality of potential effects, generate a ranking of the plurality of constitutional events as a function of the significance level, time of occurrence, and temporal effect factor of each constitutional event, receive at least a current occurrence input from the human subject, classify the at least a current occurrence input to an identified potential effect of the plurality of potential effects as a function of the ranking, and output the identified potential effect.

### FIELD OF THE INVENTION

The present invention generally relates to the field of data analysis. In particular, the present invention is directed to systems and methods for temporally sensitive causal heuristics.

### BACKGROUND

It is frequently the case that a potentially damaging event can be best averted or alleviated when detected in its nascent stages. However, an incipient crisis, albeit catastrophic when mature, may be difficult to separate from the noise of meaningless occurrences, or to distinguish from phenomena destined to remain inconsequential. Thus, early detection eludes even sophisticated analytical systems.

### SUMMARY OF THE DISCLOSURE

In an aspect, a system for temporally sensitive causal heuristics, the system comprising a computing device includes a computing device configured to provide a plurality of constitutional events and a plurality of potential effects relating to a human subject, wherein each constitutional event of the plurality of constitutional events includes an event type, a significance level, a time of occurrence, a temporal function, and at least a potential effect of the plurality of potential effects, wherein providing further includes receiving training data associating event types with temporal functions, training a temporal model using the training data, and generating the temporal function as a function of the temporal model and the event type of the constitutional event, generate a ranking of the plurality of constitutional events as a function of the significance level, time of occurrence, and temporal effect factor of each constitutional event, receive at least a current occurrence input from the human subject, classify the at least a current occurrence input to an identified potential effect of the plurality of potential effects as a function of the ranking, and output the identified potential effect.

In another aspect a method of temporally sensitive causal heuristics includes providing, by a computing device, a plurality of constitutional events and a plurality of potential effects relating to a human subject, wherein each constitutional event of the plurality of constitutional events includes an event type, a significance level, a time of occurrence, a temporal function, and at least a potential effect of the plurality of potential effects wherein providing further includes receiving training data associating event types with temporal functions, training a temporal model using the training data, and generating the temporal function as a function of the temporal model and the event type of the constitutional event. The method includes generating, by the computing device, a ranking of the plurality of constitutional events as a function of the significance level, time of occurrence, and temporal effect factor of each constitutional event. The method includes receiving, by the computing device, at least a current occurrence input from the human subject. The method includes classifying, by the computing device, the at least a current occurrence input to an identified potential effect of the plurality of potential effects as a function of the ranking. The method includes outputting, by the computing device, the identified potential effect.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for temporally sensitive causal heuristics;

FIG. 2 is a block diagram illustrating an exemplary embodiment of a machine-learning module;

FIG. 3 is a block diagram illustrating an exemplary embodiment of a human subject database;

FIG. 4 is a block diagram representing an exemplary embodiment of an expert database;

FIG. 5 is a flow diagram representing an exemplary embodiment of a method of temporally sensitive causal heuristics; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

Embodiments described in this disclosure interpret inputs describing current symptoms or other data regarding a human subject's constitution and output possible underlying causes. This may be accomplished by ranking event history and constitutional state of human subject, with associated causes related to input data, to generate a heuristic enabling rapid determination of potential causes. This may enable human subject to assess potential hazards or to detect maladies at earlier stages than have heretofore been typical.

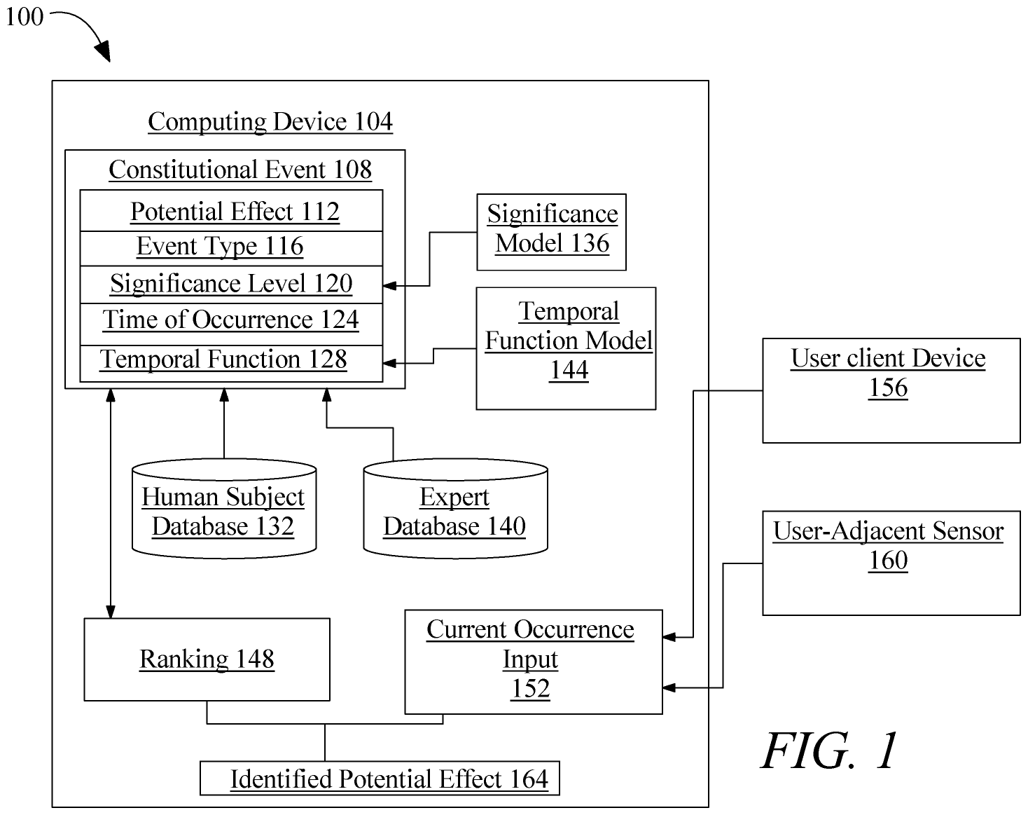

Referring now to FIG. 1, an exemplary embodiment of a system 100 for temporally sensitive causal heuristics is illustrated. System includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device 104 operating independently or may include two or more computing device 104 operating in concert, in parallel, sequentially or the like; two or more computing devices may be included together in a single computing device 104 or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices in a first location and a second computing device 104 or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device 104, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device 104.

Computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 is designed and configured to provide a plurality of constitutional events 108 relating to a human subject. A “constitutional event 108,” as used in this disclosure, is a data structure describing an event having a detected and/or potential effect 112 on a human subject state of health. Examples of events described by constitutional events 108 may include accidents such as accidental falls, car crashes, or the like, illnesses, medical procedures, physiological changes such as significant gained or lost weight, diagnosis of one or more chronic and/or degenerative conditions, or any other examples that may occur to persons skilled in the art, upon reviewing the entirety of this disclosure. Each constitutional event 108 may include at least a potential effect 112. A “potential effect” as defined in this disclosure, is an element of data describing a symptom or other measurable effect on health of a user that may result from an event described in a constitutional event 108. Each potential effect 112 may be represented as any suitable data structure; for instance, and without limitation a constitutional event 108, an identifier thereof, and/or a link thereto. For instance, and without limitation, a potential effect 112 of a car accident may include a concussion, a subdural hematoma, a spinal injury, a soft-tissue injury, an injury to an internal organ such as the spleen, or the like. A potential effect 112 of a recent or ongoing bout of influenza may include Guillain-Barre Syndrome, Kawasaki Syndrome, pneumonia, and/or Reye's Syndrome. A potential effect 112 of mononucleosis may include hepatitis. In addition to potential effects 112 associated with constitutional events 108, computing device 104 may have access to a plurality of potential effects 112 such as bacterial or viral infections, which may have associated probabilities of occurrence; such potential effects 112 may have other elements of constitutional events 108 as described below, and may be ranked with constitutional events 108 as described below. Probability of occurrence may be computed using machine-learning processes as described below, for instance by processes as described below for detection of latent constitutional events 108 and/or using training data, such as training data received from experts, associating potential effects 112 with probabilities and/or relative frequencies of occurrence within a population, which may be a population selected using a classifier as described below in further detail.

Further referring to FIG. 1, each constitutional event 108 of the plurality of constitutional events 108 includes an event type 116 An “event type 116,” as used in this disclosure, is an element of textual data identifying an event represented by a constitutional event 108; event types 116 may describe overlapping events, such as generic events (e.g. “infection,” “bacterial infection,” or “viral infection”), which may overlap with and/or include specific events (e.g., “staphylococcus infection,” “streptococcus infection,” “severe acute respiratory syndrome coronavirus 2 infection”), such that system 100 may select a more specific potential effect 112 where possible, and a more generic potential effect 112 where specificity is not available. Each constitutional event 108 of the plurality of constitutional events 108 includes a significance level 120, where a “significance level” is defined as a quantitative value indicative of a degree to which a constitutional event 108 is likely to be a causative agent in potential effects 112 experienced and/or suffered by human subject and/or a degree to which constitutional event 108 is likely to result in a serious or life-threatening potential effect 112; significance level 120 may be a product of probability of causation of a potential effect 112 and severity of possible effects, a linear combination created by multiplying a severity of each potential effect 112 of the constitutional event 108 by the probability of occurrence of the potential effect 112, summed, averaged, or otherwise combined together. Each constitutional event 108 of the plurality of constitutional events 108 includes a time of occurrence 124 defined as a timestamp indicating recorded and/or estimated time of onset, such as a time of a car crash, an estimated time of infection, or the like. Each constitutional event 108 of the plurality of constitutional events 108 includes a temporal function, defined as a numerical quantity and/or function indicating a degree to which a given constitutional event 108 will increase and/or decrease in significance over time; for instance, a degenerative disease such as multiple sclerosis or Huntington's disease may have a temporal function indicating an increase in significance over time, where increase may be linear, polynomial, exponential, or the like, a chronic stable condition may have a temporal function indicating more or less constant significance, and an acute event that fades over time such as a car crash or infection, may have a temporal function that causes significance to reduce gradually or precipitously. Computing device 104 may be configured to determine one or more elements of a constitutional event 108 using a machine-learning process. Each constitutional event 108 of plurality of constitutional events 108 may be stored in a data store such as without limitation a human subject database.

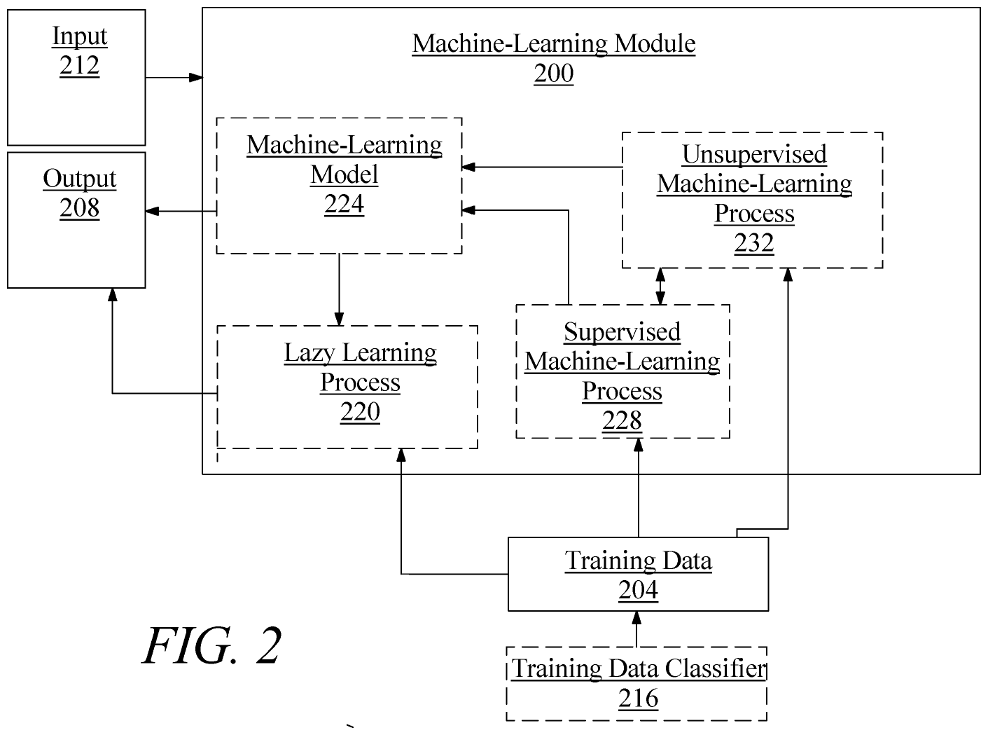

Referring now to FIG. 2, an exemplary embodiment of a machine-learning module 200 that may perform one or more machine-learning processes as described in this disclosure, is illustrated. Machine-learning module may include any suitable Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm that will be performed by a computing device 104/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a processes whereby a computing device 104 and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to a cohort of persons and/or users having similar health and/or demographic characteristics to human subject.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model 224,” as used in this disclosure, is a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may inputs as described in this disclosure as inputs, outputs as described in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 2, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using training data 204.

Referring again to FIG. 1, computing device 104 may be configured to generate, for a constitutional event 108 of the plurality of constitutional events 108, significance level 120 of the constitutional event 108 using a machine-learning module and/or process as described above. For instance, generating a significance level 120 may include receiving training data associating event types 116 with significance levels 120. Training data may be received, without limitation, as a plurality of expert inputs, which may be stored in an expert database 140; training data may be limited to records classified to user as described above. Generating significance level 120 may include training a significance model 136 as a function of the training data; training may be performed using any machine-learning process as described above. Computing device 104 may generate significance level 120 as a function of the event type 116 of the constitutional event 108 and the significance model 136, for instance by inputting event type 116 and outputting, from the significance model 136, the significance level 120.

With continued reference to FIG. 1, computing device 104 may be configured to generate, for a constitutional event 108 of the plurality of constitutional events 108, a temporal function of the constitutional event 108. Generating may include receiving training data associating event types 116 with temporal functions. Training data may be received, without limitation, as a plurality of expert inputs, which may be stored in an expert database 140; training data may be limited to records classified to user as described above. Generating may include training a temporal function model 144 as a function of the training data; training may be performed using any machine-learning process as described above. Computing device 104 may be configured to generate temporal function as a function of the temporal factor model 144 and an event type 116 of the constitutional event 108, for instance by inputting event type 116 and outputting, from the temporal model, the temporal function.

Still referring to FIG. 1, constitutional events 108 of plurality of constitutional events 108 may include at least a confirmed event. A “confirmed event,” as used in this disclosure, is a constitutional event 108 that has been entered in system by an explicit instruction identifying the associated event, such as an entry of a test result, entry indicating diagnosis by a medical professional, or the like

Alternatively or additionally, and further referring to FIG. 1, constitutional events 108 of plurality of constitutional events 108 may include at least a latent event. A “latent event,” as defined in this disclosure, is a constitutional event 108 corresponding to a medical condition that is not yet diagnosed but is probable given biological extraction data of human subject. A biological extraction, as used in this disclosure, is an element of physiological data associated with human subject, and may include any biological extraction as described in U.S. Nonprovisional application Ser. No. 16/659,817, filed on Oct. 22, 2019, and entitled “METHODS AND SYSTEMS FOR IDENTIFYING COMPATIBLE MEAL OPTIONS,” the entirety of which is incorporated herein by reference. Determination of a latent event may be performed according to any process or process step for determination of a prognostic label as described in U.S. Nonprovisional application Ser. No. 16/372,512, filed on Apr. 2, 2019, and entitled “METHODS AND SYSTEMS FOR UTILIZING DIAGNOSTICS FOR INFORMED VIBRANT CONSTITUTIONAL GUIDANCE,” the entirety of which is incorporated herein by reference

With continued reference to FIG. 1, computing device 104 is configured to generate a ranking 148 of the plurality of constitutional events 108. A “ranking,” as used in this disclosure, is a placement in an ascending or descending numerical order of constitutional events 108, where numerical order may be a numerical order of quantities, referred to for this purpose as “ranks” associated with each constitutional event 108. A rank may be calculated for each constitutional event 108 as a function of a significance level 120, time of occurrence 124, and temporal effect factor of each constitutional event 108. For instance, significance level 120 may be used as an initial rank of a constitutional event 108, and may be multiplied with or otherwise modified by an output, which may be termed a “temporal factor 128,” of a temporal function of the constitutional event 108, computed using time elapsed since time of occurrence 124; this may have an effect of increasing with the passage of time the significance of events that become more significant over time, such as progressive and/or degenerative illnesses, while decreasing with the passage of time the significance of events that become less significant over time, as predicted by temporal functions. For instance, a user who has been in a car crash a day ago may be far more likely to suffer complications such as subdural hematoma than one who suffered the car crash a week ago. In an embodiment, ranking 148 of constitutional events 108 may be combined with a ranking of potential effects 112 not connected to constitutional events 108, which may be ranked according to severity and probability, and added to ranking 148 in the form of additional constitutional events 108; probability may depend on prognostic determinations from user biological extraction, age, demographics, or the like.

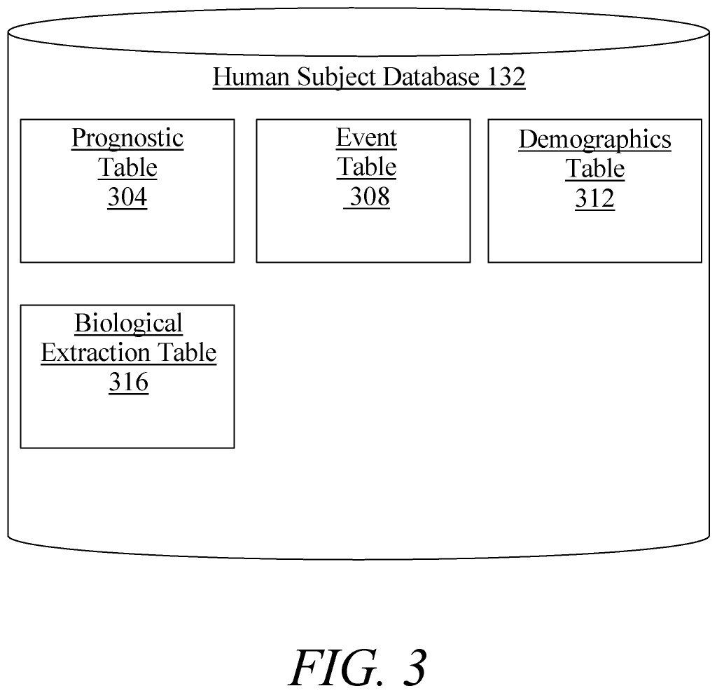

Referring now to FIG. 3, an exemplary embodiment of a human subject database 132 is illustrated. Human subject database 132 may be implemented, without limitation, as a relational database, a key-value retrieval database such as a NOSQL database, or any other format or structure for use as a database that a person skilled in the art would recognize as suitable upon review of the entirety of this disclosure. Human subject database 132 may alternatively or additionally be implemented using a distributed data storage protocol and/or data structure, such as a distributed hash table or the like. Human subject database 132 may include a plurality of data entries and/or records as described above. Data entries in a human subject database 132 may be flagged with or linked to one or more additional elements of information, which may be reflected in data entry cells and/or in linked tables such as tables related by one or more indices in a relational human subject database 132. Persons skilled in the art, upon reviewing the entirety of this disclosure, will be aware of various ways in which data entries in a human subject database 132 may store, retrieve, organize, and/or reflect data and/or records as used herein, as well as categories and/or populations of data consistently with this disclosure.

Still referring to FIG. 3, human subject database 132 may include a prognostic table 304 which may list latent events and/or other determinations of current and/or likely medical conditions of user based on biological extraction. Human subject database 132 may include an event table 308 which may list constitutional events 108. Human subject database 132 may include a demographics table 312 which may list one or more demographic data concerning human subject; demographic data may be used, without limitation, to classify training data as described above in reference to FIG. 2. Human subject database 132 may include a biological extraction table 316 which may list one or more biological extraction data concerning human subject; biological extraction data may be used, without limitation, to classify training data as described above in reference to FIG. 2. Persons skilled in the art, upon reviewing the entirety of this disclosure, will be aware of various additional or alternative data and/or tables that may be maintained in human subject database 132.

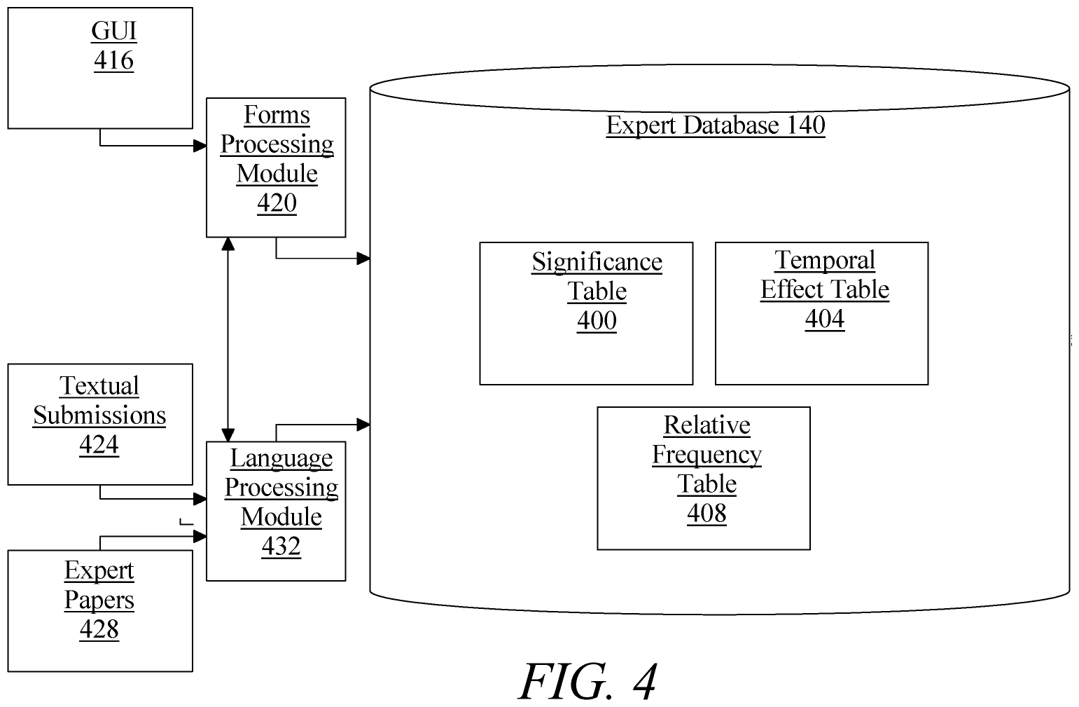

Referring now to FIG. 4, an exemplary embodiment of an expert database 140 is illustrated. Expert database 140 may have any form suitable for use as human subject database 132. Expert database 140 may, as a non-limiting example, organize data stored in the expert database 140 according to one or more database tables. One or more database tables may be linked to one another by, for instance, common column values. For instance, a common column between two tables of expert database 140 may include an identifier of an expert submission, such as a form entry, textual submission, expert paper, or the like, for instance as defined below; as a result, a query may be able to retrieve all rows from any table pertaining to a given submission or set thereof. Other columns may include any other category usable for organization or subdivision of expert data, including types of expert data, names and/or identifiers of experts submitting the data, times of submission, or the like; persons skilled in the art, upon reviewing the entirety of this disclosure, will be aware of various ways in which expert data from one or more tables may be linked and/or related to expert data in one or more other tables.

Still referring to FIG. 4, one or more database tables in expert database 140 may include, as a non-limiting example, a significance table 400. Significance table 400 may list significance of each constitutional event 108 and/or quantities suitable for calculation thereof, as reported by experts and/or as included in training data therefor. For instance, and without limitation, columns listed in disease impact table may correspond to net effect on life expectancy, degree of disability, age of onset, frequency within population, and/or other elements suitable for use in calculation of significance score. One or more database tables in expert database 140 may include, as a non-limiting example, a temporal effect table 404. Temporal effect table 404 may contain entries associating each event with temporal functions as input by experts. One or more database tables in expert database 140 may include, as a non-limiting example, a relative frequency table 408, which may list expert entries describing relative frequency within one or more populations of potential effects 112 and/or events described in constitutional events 108, where populations may be any population selectable using a classifier of training data as described above in reference to FIG. 2.

In an embodiment, and still referring to FIG. 4, a forms processing module 420 may sort data entered in a submission via a graphical user interface 416 receiving expert submissions by, for instance, sorting data from entries in the graphical user interface 416 to related categories of data; for instance, data entered in an entry relating in the graphical user interface 416 to significance may be sorted into variables and/or data structures for impact score data, which may be provided to significance table 400, while data entered in an entry relating to temporal effects on events disease may be sorted into variables and/or data structures for the storage of such data, such as temporal effect 404, relative frequencies may be sorted to relative frequency table 408, and the like. Where data is chosen by an expert from pre-selected entries such as drop-down lists, data may be stored directly; where data is entered in textual form, a language processing module may be used to map data to an appropriate existing label, for instance using a vector similarity test or other synonym-sensitive language processing test to map data to existing labels and/or categories. Similarly, data from an expert textual submissions 424, such as accomplished by filling out a paper or PDF form and/or submitting narrative information, may likewise be processed using language processing module, which may be implemented, without limitation as described in U.S. Nonprovisional application Ser. No. 16/372,512.

Data may be extracted from expert papers 428, which may include without limitation publications in medical and/or scientific journals, by language processing module 432 via any suitable process as described herein. Persons skilled in the art, upon reviewing the entirety of this disclosure, will be aware of various additional methods whereby novel terms may be separated from already-classified terms and/or synonyms therefore, as consistent with this disclosure.

Referring again to FIG. 1, computing device 104 is configured to receive at least a current occurrence input 152 from the human subject. A “current occurrence input 152,” as used in this disclosure, is an element of data describing a symptom of a user, including without limitation a change in heart rate, sleeping patterns, activity level, appetite, or the like, a sensation of pain, dizziness, drowsiness, confusion, or the like, changes in coloration of one or more body parts, or any other symptomatic description that may occur to persons skilled in the art, upon reviewing the entirety of this disclosure. Each current occurrence input 152 may include and/or be associated with a timestamp indicating time of entry in system; each input may alternatively or additionally include a timestamp for time of inception of current occurrence event, such as without limitation a user estimate of time of inception. Receipt of current occurrence event “from” human subject, in this context, refers to receipt concerning human subject, such a entry by human subject in a graphical user interface or other facility using a client device operated by human subject, entry by another person observing human subject, and/or receipt of data from a user-adjacent sensor 160. A “user-adjacent sensor 160,” as used in this disclosure, includes a sensor that detects data and/or symptoms of human subject, including without limitation a wearable device. User-adjacent sensor 160 may include a motion sensor such as without limitation a fitness activity monitor, a heart rate monitor, breath monitor, a device used to track sleep patterns, and/or any other device that may occur to persons skilled in the art upon reviewing the entirety of this disclosure.

Further referring to FIG. 1, computing device 104 is configured to classify at least a current occurrence input 152 to an identified potential effect 164 of plurality of potential effects 112 as a function of ranking 148. As a non-limiting example, computing device 104 may be configured to classify at least a current occurrence input 152 to an identified potential effect 164 of the plurality of potential effects 112 by calculating a distance metric from the at least a current occurrence input 152 to each potential effect 112 of the plurality of potential effect 112. Distance metric may include any distance metric usable in a classification process. For instance, a classifier may be configured to output at least a datum that labels or otherwise identifies a set of data that are clustered together, found to be close under a distance metric as described below, or the like. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σrᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Computing device 104 may further weight distance metric by ranking of corresponding constitutional events 108. Weighting may include, without limitation, inverse weighting, defined as multiplication by a reciprocal of ranking so that a higher rank corresponds to a lowered distance metric. Similarly, where weighting is performed using relative frequency, severity, or any other element herein, weighting may be performed using inverse weighting. In an embodiment, potential effect 112 may alternatively or additionally be weighted alone and/or per constitutional event 108, for instance by severity and probability, and then weighted by ranking of event if associated. Where two constitutional events 108 relate to an identical potential effect 112, weighted entries may be combined, for instance by addition. Computing device 104 may determine that identified potential effect 164 minimizes the weighted distance metric, or otherwise is a most likely match according to any classification process as described above. As result of classification may include a mixture of likely causes for symptoms of a user, as predicted by relative frequency and/or event history, from which a selected cause, corresponding to identified potential effect 164, is chosen by processes described above to identify the most urgent and likely possible cause; as a result, human subject and/or another user interacting therewith such as a doctor, nurse, nurse practitioner, aide, or other medical professional, may check for such causes and either confirm or eliminate them. As a result, potential causes may be investigated in order of greatest potential for impact on human subject.

Continuing to refer to FIG. 1, computing device 104 is configured to output identified potential effect 164. Output may be performed according to any suitable method, including display on a user client device 160 operated by human subject, a medical professional, and/or another person.

In an embodiment, and further referring to FIG. 1, computing device 104 may receive an input indicating that identified potential effect 164 is incorrect. For instance, a medical professional may perform a test or diagnostic procedure, human subject or another user may perform a home test, or an automated test may be performed, eliminating identified potential effect 164 and/or indicating its probability is below a preconfigured threshold number. System may remove potential effect 112 and select another potential effect 112 using any method for classification to potential effect 112 as described above. This may be performed iteratively, as human subject, medical professional, and/or other agent traverses potential effects 112 in order of selection until finding one that can be confirmed as actually occurring and/or traversing the entire list; in the latter case, system may recommend further testing and/or may indicate that current effect is likely harmless.

With continued reference to FIG. 1, system may receive another current occurrence, according to any process described above including without limitation administration of medical tests, combine current occurrence with previously received current occurrences, and perform classification a second time to identify a potential effect 112. This may also be performed iteratively, and iterations may, for instance, be driven by further user inputs in response to questions posted to user, and/or further inputs received from user-adjacent sensor 160. Questions posted to user, and/or prompts requesting further information, may be generated using, for instance, expert entries describing information useful for diagnosis of a potential effect 112, which may be stored, without limitation, in expert database 140 and converted to prompts for user data entry.

Still referring to FIG. 1, computing device 104 may be configured to receive a confirmation of the identified potential effect 164. A “confirmation,” as used in this description, is an entry explicitly confirming that a potential effect 112 is occurring in human subject; confirmation may be entered by a medical professional and/or generated automatically upon a positive output from a test that is dispositive in nature, such as a blood sugar level, automated detection of a toxin, automated detection of an infectious agent, or the like. Confirmation may be accompanied with specifics; for instance, where a more general identified potential effect 164 was first identified, a medical professional may evaluate human subject for one or more possibilities within general identified potential effect 164 and may enter a more specific example. Alternatively or additionally, a medical professional or another person may enter a more specific diagnosis, which may replace the generalized one. Computing device 104 may be configured to generate a new constitutional event 108 as a function of the identified potential effect 164. Generation may include any or all processes and/or process steps described above, including without limitation classification and/or machine learning, as well as entry of event type 116 and/or selection thereof. Time of event may be calculated as a time of detection and/or inception at least a current occurrence, as described above, and/or may be entered by a medical professional. In the latter case, entry may be informed by time of at least current occurrence, which may be displayed and thus available for use in determining a timeline. Computing device 104 may add new constitutional event 108 to the plurality of constitutional events 108. Computing device 104 is may be configured to re-generate the ranking, according to any process and/or process step described above. Subsequent occurrences may be evaluated using the regenerated ranking; above described process steps may be iterated an indefinite number of times.

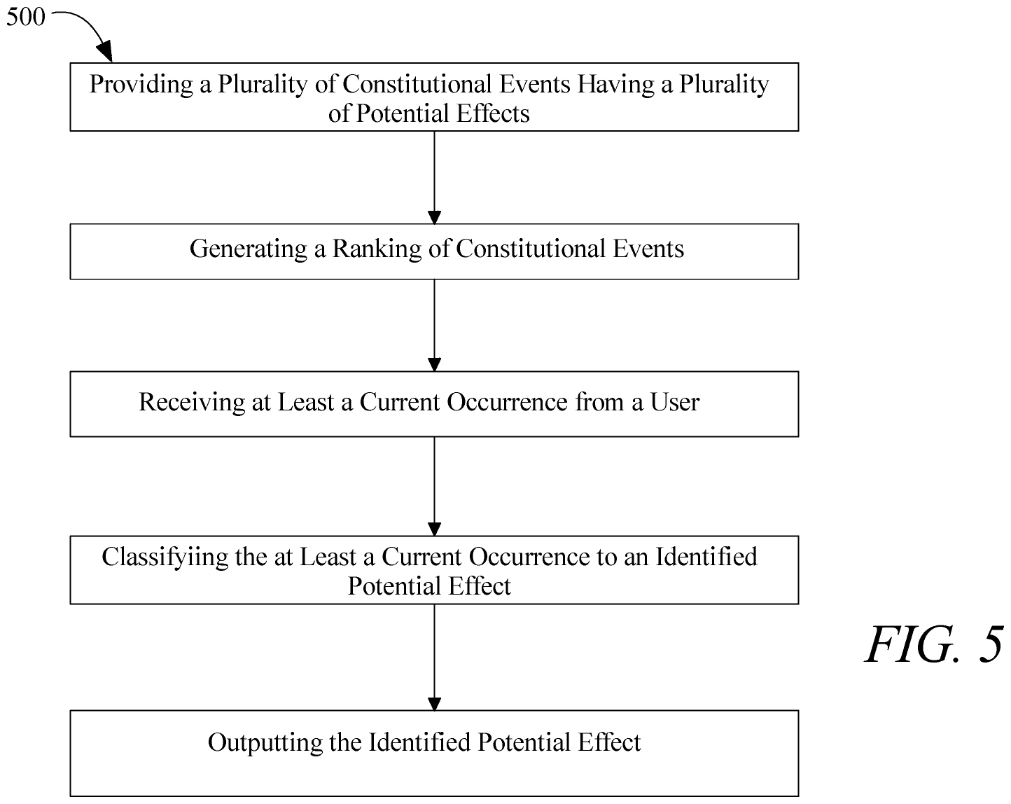

Referring now to FIG. 5, an exemplary embodiment of a method of temporally sensitive causal heuristics is illustrated. At step 505, a computing device 104 provides a plurality of constitutional events 108 having a plurality of potential effects 112 relating to a human subject, wherein each constitutional event 108 of the plurality of constitutional events 108 includes an event type 116, a significance level 120, a time of occurrence 124, a temporal function, and at least a potential effect 112 of the plurality of potential effects 112; this may be implemented, without limitation, as described above in reference to FIGS. 1-4. Computing device 104 may generate, for one or more constitutional events 108 of the plurality of constitutional events 108, the significance level 120 of the constitutional event 108. Generating may include receiving training data associating event types 116 with significance levels 120, training a significance model 136, and generating significance level 120 as a function of the event type 116 of the constitutional event 108 and the significance model 136. Computing device 104 may generate, for one or more constitutional events 108 of the plurality of constitutional events 108, the temporal function of the constitutional event 108; generating may include receiving training data associating event types 116 with temporal functions, training a temporal model, and generating the temporal function as a function of the temporal model and the event type 116 of the constitutional event 108. Plurality of constitutional events 108 further includes at least a confirmed event. Plurality of constitutional events 108 further includes at least a latent event.

At step 510, and still referring to FIG. 5, computing device 104 generates a ranking of the plurality of constitutional events 108 as a function of the significance level 120, time of occurrence 124, and temporal effect factor of each constitutional event 108; this may be implemented, without limitation, as described above in reference to FIGS. 1-4.

At step 515, and with continued reference to FIG. 5, computing device 104 receives at least a current occurrence input 152 from the human subject; this may be implemented, without limitation, as described above in reference to FIGS. 1-4. Receiving at least a current occurrence input 152 may include receiving the at least a current occurrence input 152 by receiving at least a user entry. Receiving at least a current occurrence input 152 may include receiving the at least a current occurrence input 152 by receiving a transmission from a user-adjacent sensor 160.

At step 520, computing device 104 classifies at least a current occurrence input 152 to an identified potential effect 164 of the plurality of potential effects 112 as a function of the ranking; this may be implemented, without limitation, as described above in reference to FIGS. 1-4. Classifying at least a current occurrence input 152 to an identified potential effect 164 of the plurality of potential effects 112 may include calculating a distance metric from the at least a current occurrence input 152 to each potential effect 112 of the plurality of potential effect 112, weighting the distance metric by the ranking of corresponding constitutional events 108, and determining that the identified potential effect 164 minimizes the weighted distance metric.

At step 525, computing device 104 outputs identified potential effect 164; this may be implemented, without limitation, as described above in reference to FIGS. 1-4. Computing device 104 may receive a confirmation of the identified potential effect 164, generate a new constitutional event 108 as a function of the identified potential effect 164, and add the new constitutional event 108 to the plurality of constitutional events 108. Computing device 104 may re-generate the ranking.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

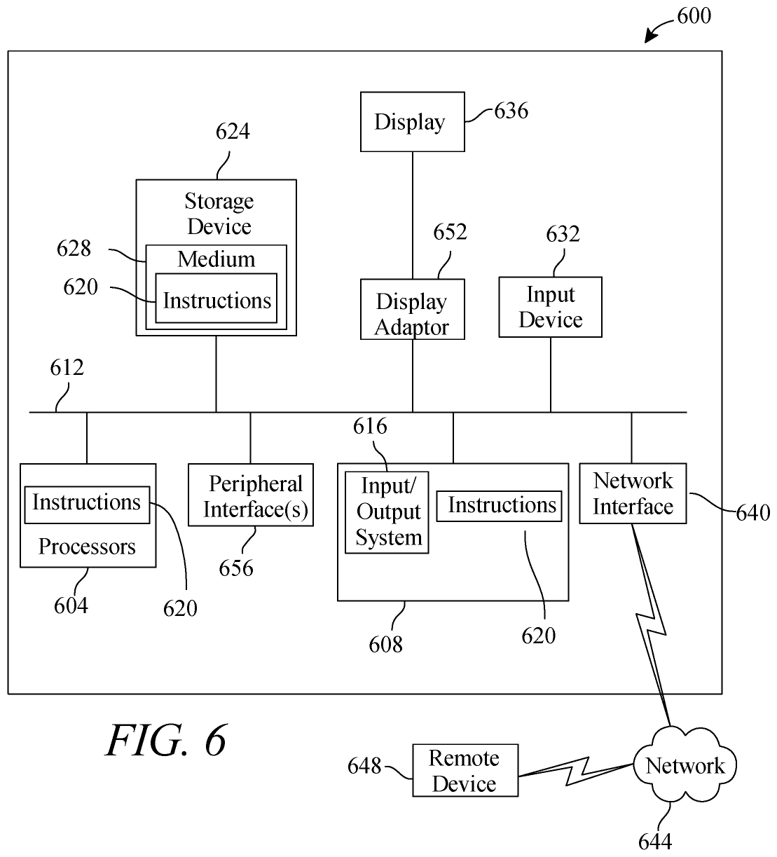

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Processor 604 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 604 may be organized according to Von Neumann and/or Harvard architecture as a non-limiting example. Processor 604 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 608 may include various components (e.g., machine-readable media) including, but not limited to, a random-access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 600 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display 636, discussed further below. Input device 632 may be utilized as a user selection device for selecting one or more graphical representations in a graphical interface as described above.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote devices 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for temporally sensitive causal heuristics, the system comprising a computing device, the computing device designed and configured to:
provide a plurality of constitutional events and a plurality of potential effects relating to a human subject, wherein each constitutional event of the plurality of constitutional events includes an event type, a significance level, a time of occurrence, a temporal function, and at least a potential effect of the plurality of potential effects, wherein providing further comprises:
receiving training data associating event types with temporal functions;
training a temporal model using the training data; and
generating the temporal function as a function of the temporal model and the event type of the constitutional event;
generate a ranking of the plurality of constitutional events as a function of the significance level, time of occurrence, and temporal effect factor of each constitutional event;
receive at least a current occurrence input from the human subject;
classify the at least a current occurrence input to an identified potential effect of the plurality of potential effects as a function of the ranking; and
output the identified potential effect.

2. The system of claim 1, wherein the computing device is further configured to generate, for a constitutional event of the plurality of constitutional events, the significance level of the constitutional event, wherein generating further comprises:
receiving training data associating event types with significance levels;
training a significance model using the training data; and
generating the significance level as a function of the event type of the constitutional event and the significance model.

3. The system of claim 1, wherein the plurality of constitutional events further includes at least a confirmed event.

4. The system of claim 1, wherein the plurality of constitutional events further includes at least a latent event.

5. The system of claim 1, wherein the computing device is configured to receive the at least a current occurrence input from the human subject by receiving at least a user entry.

6. The system of claim 1, wherein the computing device is configured to receive the at least a current occurrence input from the human subject by receiving a transmission from a user-adjacent sensor.

7. The system of claim 1, wherein the computing device is further configured to classify at least a current occurrence input to an identified potential effect of the plurality of potential effects by:
calculating a distance metric from the at least a current occurrence input to each potential effect of the plurality of potential effect;
weighting the distance metric by the ranking of corresponding constitutional events; and
determining that the identified potential effect minimizes the weighted distance metric.

8. The system of claim 1, wherein the computing device is further configured to:
receive a confirmation of the identified potential effect;
generate a new constitutional event as a function of the identified potential effect; and
add the new constitutional event to the plurality of constitutional events.

9. The system of claim 8, wherein the computing device is further configured to re-generate the ranking.

10. The system of claim 1, wherein the computing device is further configured to:
receive an input indicating that the identified potential effect is incorrect;
remove the identified potential effect; and
select an alternative potential effect from the plurality of potential effects.

11. A method of temporally sensitive causal heuristics, the method comprising:
providing, by a computing device, a plurality of constitutional events and a plurality of potential effects relating to a human subject, wherein each constitutional event of the plurality of constitutional events includes an event type, a significance level, a time of occurrence, a temporal function, and at least a potential effect of the plurality of potential effects, wherein providing further comprises:
receiving training data associating event types with temporal functions;
training a temporal model using the training data; and
generating the temporal function as a function of the temporal model and the event type of the constitutional event;
generating, by the computing device, a ranking of the plurality of constitutional events as a function of the significance level, time of occurrence, and temporal effect factor of each constitutional event;
receiving, by the computing device, at least a current occurrence input from the human subject;
classifying, by the computing device, the at least a current occurrence input to an identified potential effect of the plurality of potential effects as a function of the ranking; and
outputting, by the computing device, the identified potential effect.

12. The method of claim 11 further comprising generating, for a constitutional event of the plurality of constitutional events, the significance level of the constitutional event, wherein generating further comprises:
receiving training data associating event types with significance levels;
training a significance model using the training data; and
generating the significance level as a function of the event type of the constitutional event and the significance model.

13. The method of claim 11, wherein the plurality of constitutional events further includes at least a confirmed event.

14. The method of claim 11, wherein the plurality of constitutional events further includes at least a latent event.

15. The method of claim 11, wherein receiving the at least a current occurrence input further comprises receiving the at least a current occurrence input by receiving at least a user entry.

16. The method of claim 11, wherein receiving the at least a current occurrence input further comprises receiving the at least a current occurrence input by receiving a transmission from a user-adjacent sensor.

17. The method of claim 11, wherein classifying the at least a current occurrence input to an identified potential effect of the plurality of potential effects further comprises:
calculating a distance metric from the at least a current occurrence input to each potential effect of the plurality of potential effect;
weighting the distance metric by the ranking of corresponding constitutional events; and
determining that the identified potential effect minimizes the weighted distance metric.

18. The method of claim 1, further comprising:
receiving a confirmation of the identified potential effect;
generating a new constitutional event as a function of the identified potential effect; and
adding the new constitutional event to the plurality of constitutional events.

19. The method of claim 19 further comprising: re-generate the ranking.

20. The method of claim 1, further comprising:
receiving an input indicating that the identified potential effect is incorrect;
removing the identified potential effect; and
selecting an alternative potential effect from the plurality of potential effects.
